(19) United States
(12) Patent Application Publication
(10) Pub. No.: US 2025/0232093 A1
(43) Pub. Date: Jul. 17, 2025

(54) MULTI-LABEL NEURAL ARCHITECTURE FOR MODELING DNA-ENCODED LIBRARIES DATA

(71) Applicant: GOOGLE LLC, Mountain View, CA (US)

(72) Inventors: Kehang HAN, Mountain View, CA (US); Steven KEARNES, Mountain View, CA (US); Jin XU, Mountain View, CA (US)

(21) Appl. No.: 18/700,410

(22) PCT Filed: Oct. 20, 2022
(86) PCT No.: PCT/US2022/047237

§ 371 Date: Apr. 11, 2024

Related U.S. Application Data
(60) Provisional application No. 63/270,446, filed on Oct. 21, 2021.

(51) Int. Cl. G06F 30/27; G16B 15/30; G16B 35/00; G16B 40/20

(52) U.S. Cl. CPC G06F 30/27; G16B 15/30; G16B 35/00; G16B 40/20

(57) ABSTRACT

Systems and methods are provided for using DMA-encoded library experimental data to tram graph neural networks or other deep learning machine learning models to predicts the efficacy of candidate molecules (as represented by a chemical structure graph or other input indicative of the candidate's molecular structure) at binding with various target and non-target (e.g., experimental substrate materials, competitive targets) substances (e.g., receptors, proteins, specified binding sites thereof). These embodiments include training a model to separately predict the binding efficacy of an candidate substance with each of a target substance (e.g., receptor, N protein), an experimental substrate, and one or more anti-targets (e.g., competitive receptors the binding to which can cause side effects, p alternative binding sites of a target receptor that negatively impact the behavior of the receptor).

This application claims priority to U.S. Provisional Patent Application No. 63/270,446, filed Oct. 21, 2021, the contents of which are incorporated by reference.

### BACKGROUND

Unless otherwise indicated herein, the materials described in this section are not prior art to the claims in this application and are not admitted to be prior art by inclusion in this section.

The process of drug discovery is complex and expensive, and involves assessing many thousands or millions of potential drug candidates with respect to overall therapeutic efficacy. This can include experimentally assessing the efficacy with which each candidate molecule binds to a target (e.g., a target binding site of a target receptor or protein) and/or to other substances (e.g., to an experimental substrate to act as a control, to one or more “anti-targets” to which it is preferred that the candidate not bind, e.g., receptors or proteins to which binding is likely to cause negative side effects). Once screened in this manner, the best candidate molecules (e.g., those that exhibited sufficiently high binding efficacy with respect to the target while also exhibiting sufficiently low binding efficacy with respect to the experimental substrate, anti-target, etc.) can be advanced to subsequent stages of screening or development (e.g., testing in animals or cell culture, development and assessment of derivative DEL libraries based on the structure of the best candidate molecules).

High-throughput screen, DNA-encoded libraries, laboratory automation, and other techniques have reduced the cost of assessing large numbers of candidate molecules. However, such assessment remains expensive, especially for diseases/targets for which there is little information as regards potential lead compounds or structures. In some examples, predictive models have been developed to attempt to predict the efficacy of candidate molecules/structures prior to experimental assessment, in order to reduce the cost of drug discovery.

One barrier to applying machine learning (e.g., deep learning) to such drug discovery problems is limited data. The recent advent of DNA-encoded libraries (DELs) with their massive data size facilitates the training of large deep learning molecular property models. Such models can then be applied to key stages (e.g., hit-finding, hit to lead) of the drug discovery process.

The readout of such a DEL experiment is DNA sequence counts, which are commonly aggregated into disynthon representations to calculate enrichment scores with good signal-to-noise properties. Besides the primary DEL selection experiment (measuring binding signal called, e.g., “Target Enr” when protein target is present, i.e., an on-target binding experiment), control selection (measuring binding signal called, e.g., “NTC Enr” when no protein target is present, i.e., a No-Target Control binding experiment) and additional counter-selections could be run to further reduce noise, for example, inclusion of a known competitive inhibitor (measuring binding signal called, e.g., “Competitor Enr” when a protein target is competitively inhibited).

Data from such multiple DEL selections can be combined to create mutually exclusive labels corresponding to different experimental outcomes. These labels represent logical combinations (e.g. AND/OR) of enrichment scores from the multiple experimental conditions as well as external side information such as hit frequency across targets denoted as, e.g., “Target ratio” (to identify promiscuous compounds that are unlikely to have specific interactions with the target). The label derivation can be often summarized in a decision tree, which can be referred to as a column scheme. A classification model can then be built with the derived classes. One major problem with this approach is that in practice a molecule being categorized as one class can still be of another class (e.g., a “promiscuous”-labeled compound can still be a target competitive hit). Additionally, using labels derived from multiple experiments, each with their own criteria and thresholds, requires such a model to learn a complicated latent structure for the human-crafted labels (which are fixed at training time).

### SUMMARY

In a first aspect, a computer-implemented method is provided that includes: (i) obtaining training data for a plurality of experimental molecules, wherein the training data comprises structural information for each experimental molecule in the plurality of experimental molecules and information from a first DNA-encoded library (DEL) experiment that is indicative of binding affinities of at least a first portion of the plurality of experimental molecules for two or more substances, wherein the two or more substances include a target and an experimental substrate; (ii) based on the training data, determining at least two multi-class labels for each experimental molecule in the plurality of experimental molecules, wherein determining at least two multi-class labels for a given experimental molecule of the plurality of experimental molecules comprises (a) determining a first multi-class label that is indicative of a degree of enrichment of the given experimental molecule in the presence of the target, and (b) determining a second multi-class label that is indicative of a degree of enrichment of the given experimental molecule in the presence of the experimental substrate; (iii) applying the training data to train a predictive model to receive, as an input, a graph representing a chemical structure of an input molecule and to generate, as an output, the at least two multi-class labels for the input molecule, wherein the predictive model comprises a graph neural network and at least two output heads, wherein each output head of the at least two output heads generates a respective one of the at least two multi-class labels as an output; and (iv) outputting the trained predictive model.

In a second aspect, a computer-implemented method is provided that includes: applying a graph representing a chemical structure of an input molecule to a trained predictive model to generate, as an output of the model, at least two outputs for the input molecule, wherein a first output of the at least two outputs is predictive of a degree of enrichment of the input molecule in the presence of a target, wherein a second output of the at least two outputs is predictive of a degree of enrichment of the input molecule in the presence of an experimental substrate, wherein the predictive model comprises a graph neural network and at least two output heads, wherein each output head of the at least two output heads generates a respective one of the at least two outputs as an output, and wherein the trained predictive model has been trained using a training dataset that includes structural information for a plurality of experimental molecules and information from a first DNA-encoded library (DEL) experiment that is indicative of binding affinities of at least a first portion of the plurality of experimental molecules for two or more substances, wherein the two or more substances include the target and the experimental substrate.

In a third aspect, a system is provided having one or more processors that are configured to perform the method of any of the first or second aspects.

In a fourth aspect, an article of manufacture is provided that includes a non-transitory computer-readable medium, having stored thereon program instructions that, upon execution by a computing device, cause the computing device to perform operations to effect the method of any of the first or second aspects.

### BRIEF DESCRIPTION OF THE DRAWINGS

FIG. 1 depicts aspects of an example system.

FIG. 2 depicts aspects of an example method.

FIG. 3 depicts aspects of an example method.

FIG. 4 depicts aspects of an example method.

FIG. 5 depicts aspects of an example experiment.

FIG. 6 depicts experimental results.

FIG. 7A depicts experimental results.

FIG. 7B depicts experimental results.

### DETAILED DESCRIPTION

In the following detailed description, reference is made to the accompanying figures, which form a part hereof In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein

### I. Overview

It is desirable to generate computational models to predict the utility of arbitrary chemical structures (e.g., small molecules) in the treatment of various diseases. Potential drug candidates can then be cheaply and quickly pre-screened by the computational model. Drug candidates that the model predicts are most likely to be effective in treating the disease can then be assessed experimentally. This can reduce the cost and time required to assess a class of candidate molecules by reducing the number of molecules within the class that are experimentally validated. This can include using DNA-encoded libraries (DEL) or other experimental processes to assess the ability of each pre-selected candidate molecule to specifically bind to a target (e.g., a receptor protein implicated in a disease process of interest) while avoiding binding to “anti-targets” (e.g., to receptor protein(s) implicated in common unwanted side effects). Such a model may receive as input a graph that is representative of a candidate molecule's chemical structure (e.g., the model could include a graph neural network) or could receive some other input that is representative of the structure of the candidate molecule and could provide one or more outputs indicative of the efficacy of candidate molecule at binding with a target while avoiding binding to one or more anti-targets, experimental substrates, etc. or some other information that may be relevant to the clinical utility of the molecule.

Such a model can be trained with a training dataset that includes information (e.g., the structure) about a plurality of different candidate molecules and information about the degree, selectivity, or other information related to the binding of each of those molecules to a target and/or to other substances (e.g., to a control substance, to one or more anti-targets or other confounding substances). In practice, such binding information can be represented as a multi-class label for each of the molecules, which represents the utility of each molecule with respect to how specifically it can bind to the target while avoiding binding to other substances in general, and to specified anti-targets and/or experimental substrate material in particular. For example, the mutually-exclusive classes that could describe each molecule in a training dataset could include “matrix binder” for molecules that are enriched by more than a threshold amount in a control experiment where experimental substrate is the only available material to bind to, “promiscuous binder” for molecules that are not “matrix binders” but that do not specifically bind to the target. “non hit” for molecules that are not “matrix binders” or “promiscuous binders” but that fail to reach a threshold degree of enrichment in binding to the target, “non competitive hit” for molecules that are not “matrix binders,” “promiscuous binders,” or “non hits” but that enrich one or more specified anti-target competitor substances by more than a threshold amount, and “competitive hit” for molecules that are not “matrix binders,” “promiscuous binders,” or “non hits” and that also do not enrich the one or more specified anti-target competitor substances by more than the threshold amount. In such an example the “competitive hit” molecules represent candidates for further investigation, as they specifically bind to the target to a sufficiently high degree while avoiding binding to the experimental substrate, the anti-targets, or to other undesired substances.

However, such an approach can require significant manual effort to specify the classes and associated thresholds and other parameters, relative to a particular set of DNA-encoded library count data or other specific experimental data. This can make it costly to use such data to train predictive models and difficult to aggregate data from different datasets (which may differ with respect to substrate material, the absolute value of relevant thresholds, etc.). Further, such mutually-exclusive classes may abolish useful data from the underlying experiments. For example, molecules labeled as “promiscuous binder” may include molecules that exhibit undesirably high affinity for one or more anti-targets along with molecules that do not exhibits such unwanted anti-target binding affinity. Thus, a predictive model trained on such a label, in addition to learning the relationships between chemical structure and efficacy at binding to a target and to other substrates, must also learn the implicit structure of the multiple classes of the label, as well as compensate for any differences that may exist in the application of the label to datasets from different experiments.

Instead, a predictive model can be trained to predict a number of different labels to describe each of the molecules. Each of the different labels can be specified to describe a more independent aspect of the activity of a molecule. For example, a first label could represent the affinity of the molecule in binding to a target molecule, a second label could represent the affinity of the molecule in binding to an experimental substrate material, a third label could represent the affinity of the molecule in binding to a specified anti-target, etc. Each label could have two classes (e.g., “enriched” and “not enriched”) or more classes (e.g., a range of ordinal classes from “minimally enriched” to “maximally enriched”), or could even be a continuous value (e.g., the predictive model could be trained to regress on one or more of the labels). The labels could be trained on enrichment values (e.g., on counts of instances of binding of a molecule to a target normalized to counts of instances of binding of the molecule in a control experiment), on raw DEL count data, or on some other DEL-derived experimental data.

Training using such a multi-label dataset provides a variety of benefits. By separating the predicted activity of the molecule with respect to each different binding substance (target, substrate, anti-target(s), etc.) into respective different output labels, the predictive model can learn to model such semi-independent properties more directly, rather than learning to model the implicit structure of a single, more complex multi-class label. Providing the training data in such a multi-label format can also have the effect of presenting the predictive model with more of the information from the experimental data, in contrast to single-label multi-class training data which can ‘hide’ some of that information (e.g., information about whether a “matrix binder” also enriches one or more specified anti-target competitor substances by more than a threshold amount).

Training on such a multi-label dataset can also reduce the amount of manual effort (e.g., in threshold setting, class definition/organization) that is required to convert experimental data (e.g., DEL experiment read counts and/or enrichment values) into class IDs or other data suitable for training the predictive model. For example, a threshold for distinguishing “enriched” from “not enriched” with respect to a particular substance (e.g., a target, an experimental substrate material, a specified anti-target) could be automatically set to a specified percentile within a dataset (e.g., molecules are labeled as “enriched” with respect to a target if they are within the top 50%, 25%, 5%, 1%, or some other specified percentile of enrichment with respect to the target within a particular experimental dataset). Such automatic labeling can also facilitate the aggregation of data from multiple different experimental datasets (e.g., from multiple different DEL experiments which may differ from each other with respect to the DNA library used, processing parameters, etc.). This is because, while the classes for a particular label may not be perfectly comparable with respect to an underlying ‘true’ enrichment level threshold, such differences are likely to be smaller (a difference of a few percent from one experiment to another) and such differences do not convolve multiple different ‘independent’ aspects of the molecules' efficacy at binding to different substances.

The use of such multi-label data to train predictive models also facilitates the use of multiple parallel output heads (e.g., associated with targets, anti-targets, substrate materials) as part of the predictive model. This allows such predictive models to be easily expanded (by the addition of output heads) to predict more outputs related to binding affinity for respective substances. This can allow such predictive models to be used to predict binding affinity for multiple anti-targets (e.g., hERG, ERa). In some examples, such an anti-target could include a specified region of the target (e.g., a specified pocket or other potential alternative binding feature of the target protein) to which binding is undesired (e.g., due to binding at that locale resulting in unwanted physiological effects and/or impeding binding to a desired primary binding region of the target). Such a model structure can also allow a trained predictive model to be used to predict the affinity of candidate molecules for additional substances (e.g., new therapeutic targets, additional anti-targets). Such a model structure can also reduce the overall computational cost and/or power required to predict the set of output labels for an input candidate chemical structure (e.g., target enrichment label, experimental substrate enrichment label, anti-target enrichment label) compared to training complete separate models for each label. This is because most of the computation involved in computing each such separate model could be represented by the computation of the in-common aspect of a multi-head model, while the computation involved in each of the individual heads can be relatively lighter-weight, as they receive relatively high-level intermediate outputs from the in-common aspect of the multi-head model.

In order to train a predictive model using such multi-label training data, the training data can be pre-processed. This can be done, e.g., to balance the number of training instances applied to the model between different label conditions. For example, training examples could be selected from a training dataset such that each multi-label condition (i.e., each possible set of classes across each of the labels) is represented by the same number of training examples or approximately the same number of training examples (e.g., the number of training examples for each multi-label condition varies by less than 5%). Where each label is a binary label (e.g., “enriched” vs. “non-enriched” with respect to each substance that corresponds to each label), the total number of training examples used could be N*2\hat{ }n, where n is the number of labels and N is the number of training examples used for each multi-label condition. Such a balancing process could include removing training examples from the training dataset and/or replicating training examples such that the replicated training examples are represented more than once in the training dataset.

Once the predictive model has been trained, it can be used to predict the efficacy of candidate molecules (which may be novel molecules not represented in the training data used to train the model). This can include applying the graph or other representation of the chemical structure of a candidate molecule to the trained model to generate outputs related to each of the multiple labels used to train the model (e.g., a first output related to the efficacy of binding of the candidate to a target, a second output related to the efficacy of binding of the candidate to an experimental substrate, a third output related to the efficacy of binding of the candidate to a first anti-target, and a fourth output related to the efficacy of binding of the candidate to a second anti-target). These model outputs could then be used to select a subset of candidate molecules for further investigation. Such further investigation could include experimental verification via an additional DEL experiment or some other experiment (e.g., a single-point inhibition experiment, a dose-response experiment), later stages of clinical assessment, or some other targeted investigation. Selecting such a subset could include using the outputs to generate an overall utility score for each of the molecules. Determining such a score could include applying a logical formula to thresholded versions of the outputs. For example, candidates for further investigations could be those molecules whose target output was greater than a first threshold, whose experimental substrate output was less than a second threshold, and whose first anti-target output was less than a third threshold. Additionally or alternatively, the score could be determined by applying a formula to continuously-valued outputs directly. For example, the score could be determined for a molecule by subtracting the molecule's experimental substrate output and first anti-target output from the molecule's target output. Such scores could then be sorted by absolute value and the top molecules (e.g., a specified number of the top molecules with respect to their overall score, a specified top percentile of the molecules with respect to their overall score). In another example, candidate molecules could be rejected based on their experimental substrate and/or anti-target outputs and the non-rejected molecules could then be sorted with respect to their target outputs in order to select a subset of candidate molecules for further investigation.

Such a single-valued overall score could also be used during training of the predictive model to inform the training. For example, such overall scores could be used to select which checkpoints and models to fold and/or replicate during model training in an ensemble model training paradigm. To do so (e.g., to select which models in the ensemble to retain), such overall scores could be compared to a validation dataset. Such a use of the overall scores could reduce the cost and/or power requirements to train the model by reducing the overall number of training iterations required to complete the training. A tuning dataset could be used to determine any tunable parameters in the formula used to determine the overall score from the model outputs (e.g., to determine the values of weights used to linearly combine the individual model outputs into the overall score).

### II. Illustrative Systems

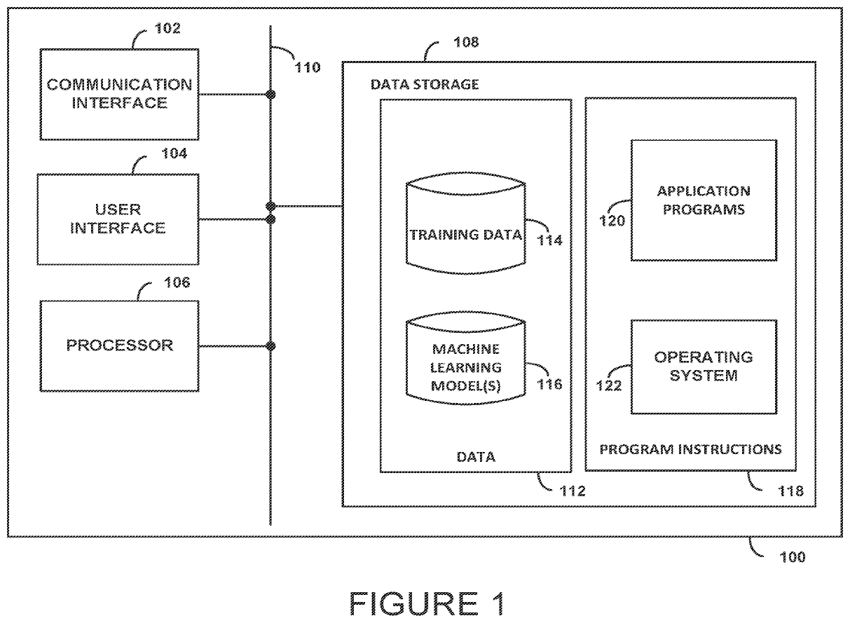

FIG. 1 illustrates an example computing system 100 that may be used to implement the methods described herein. By way of example and without limitation, computing system 100 may be a computer (such as a desktop, notebook, tablet, or handheld computer, a server), elements of a cloud computing system, or some other type of device. It should be understood that computing system 100 may represent a physical computing device such as a server, a particular physical hardware platform on which a machine learning application operates in software, or other combinations of hardware and software that are configured to carry out machine learning functions as described herein. The computing system 100 could be a central system (e.g., a server, elements of a cloud computing system) that is configured to generate and/or receive the outputs of DNA-encoded library experiments (e.g., DNA reads and/or counts) or other information (e.g., information about the binding affinity of a variety of small molecules or other candidate molecules for one or more targets, anti-targets, experimental substrates, or other substances) and to train and/or apply putative molecular structures to a predictive model as described herein.

As shown in FIG. 1, computing system 100 may include a communication interface 102, a user interface 104, a processor 106, and data storage 108, all of which may be communicatively linked together by a system bus, network, or other connection mechanism 110.

Communication interface 102 may function to allow computing system 100 to communicate, using analog or digital modulation of electric, magnetic, electromagnetic, optical, or other signals, with other devices, access networks, and/or transport networks. Thus, communication interface 102 may facilitate circuit-switched and/or packet-switched communication, such as plain old telephone service (POTS) communication and/or Internet protocol (IP) or other packetized communication. For instance, communication interface 102 may include a chipset and antenna arranged for wireless communication with a radio access network or an access point. Also, communication interface 102 may take the form of or include a wireline interface, such as an Ethernet, Universal Serial Bus (USB), or High-Definition Multimedia Interface (HDMI) port. Communication interface 102 may also take the form of or include a wireless interface, such as a Wifi, BLUETOOTH®, global positioning system (GPS), or wide-area wireless interface (e.g., WiMAX or 3GPP Long-Term Evolution (LTE)). However, other forms of physical layer interfaces and other types of standard or proprietary communication protocols may be used over communication interface 102. Furthermore, communication interface 102 may comprise multiple physical communication interfaces (e.g., a Wifi interface, a BLUETOOTH® interface, and a wide-area wireless interface).

In some embodiments, communication interface 102 may function to allow computing system 100 to communicate with other devices, remote servers, access networks, and/or transport networks. For example, communication interface 102 may function to allow computing system 100 to communicate with next-generation sequencers, automated laboratory equipment, or other apparatus configured to perform steps of a DEL experiment or other experiment for generating binding affinity data for candidate molecules against targets or other substances and/or to generate, process, and/or store outputs of such an experiment.

User interface 104 may function to allow computing system 100 to interact with a user or other entity, for example to receive input from and/or to provide output to the user. Thus, user interface 104 may include input components such as a keypad, keyboard, touch-sensitive or presence-sensitive panel, computer mouse, trackball, joystick, microphone, and so on. User interface 104 may also include one or more output components such as a display screen which, for example, may be combined with a presence-sensitive panel. The display screen may be based on CRT, LCD, and/or LED technologies, or other technologies now known or later developed. User interface 104 may also be configured to generate audible output(s), via a speaker, speaker jack, audio output port, audio output device, earphones, and/or other similar devices.

Processor 106 may comprise one or more general purpose processors—e.g., microprocessors—and/or one or more special purpose processors—e.g., digital signal processors (DSPs), graphics processing units (GPUs), floating point units (FPUs), network processors, tensor processing units (TPUs), or application-specific integrated circuits (ASICs). In some instances, special purpose processors may be capable of image processing, image alignment, merging images, transforming images, executing machine learning models, training machine learning models, among other applications or functions. Data storage 108 may include one or more volatile and/or non-volatile storage components, such as magnetic, optical, flash, or organic storage, and may be integrated in whole or in part with processor 106. Data storage 108 may include removable and/or non-removable components.

Processor 106 may be capable of executing program instructions 118 (e.g., compiled or non-compiled program logic and/or machine code) stored in data storage 108 to carry out the various functions described herein. Therefore, data storage 108 may include a non-transitory computer-readable medium, having stored thereon program instructions that, upon execution by computing system 100, cause computing system 100 to carry out any of the methods, processes, or functions disclosed in this specification and/or the accompanying drawings. The execution of program instructions 118 by processor 106 may result in processor 106 using data 112.

By way of example, program instructions 118 may include an operating system 122 (e.g., an operating system kernel, device driver(s), and/or other modules) and one or more application programs 120 (e.g., functions for executing and/or training a machine learning predictive model) installed on computing system 100. Data 112 may include training data (e.g. DNA sequence reads, counts of candidate molecule-specific DNA fragments, other data related to one or more DEL experiments, etc.) 114 and/or machine learning model(s) 116 that may be determined therefrom or obtained in some other manner.

Application programs 120 may communicate with operating system 122 through one or more application programming interfaces (APIs). These APIs may facilitate, for instance, application programs 120 transmitting or receiving information via communication interface 102, receiving and/or displaying information on user interface 104, and so on.

Application programs 120 may take the form of “apps” that could be downloadable to computing system 100 through one or more online application stores or application markets (via, e.g., the communication interface 102). However, application programs can also be installed on computing system 100 in other ways, such as via a web browser or through a physical interface (e.g., a USB port) of the computing system 100.

### III. Example Methods

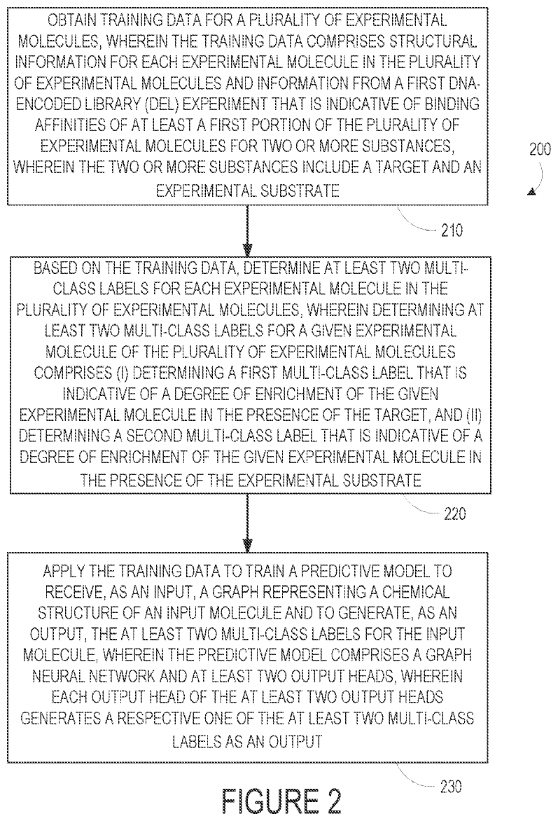

FIG. 2 is a flowchart of an example computer-implemented method 200. The method 200 includes obtaining training data for a plurality of experimental molecules, wherein the training data comprises structural information for each experimental molecule in the plurality of experimental molecules and information from a first DNA-encoded library (DEL) experiment that is indicative of binding affinities of at least a first portion of the plurality of experimental molecules for two or more substances, wherein the two or more substances include a target and an experimental substrate (210). The method 200 additionally includes, based on the training data, determining at least two multi-class labels for each experimental molecule in the plurality of experimental molecules, wherein determining at least two multi-class labels for a given experimental molecule of the plurality of experimental molecules comprises (i) determining a first multi-class label that is indicative of a degree of enrichment of the given experimental molecule in the presence of the target, and (ii) determining a second multi-class label that is indicative of a degree of enrichment of the given experimental molecule in the presence of the experimental substrate (220). The method 200 additionally includes applying the training data to train a predictive model to receive, as an input, a graph representing a chemical structure of an input molecule and to generate, as an output, the at least two multi-class labels for the input molecule, wherein the predictive model comprises a graph neural network and at least two output heads, wherein each output head of the at least two output heads generates a respective one of the at least two multi-class labels as an output (230). The method 200 could include additional or alternative features.

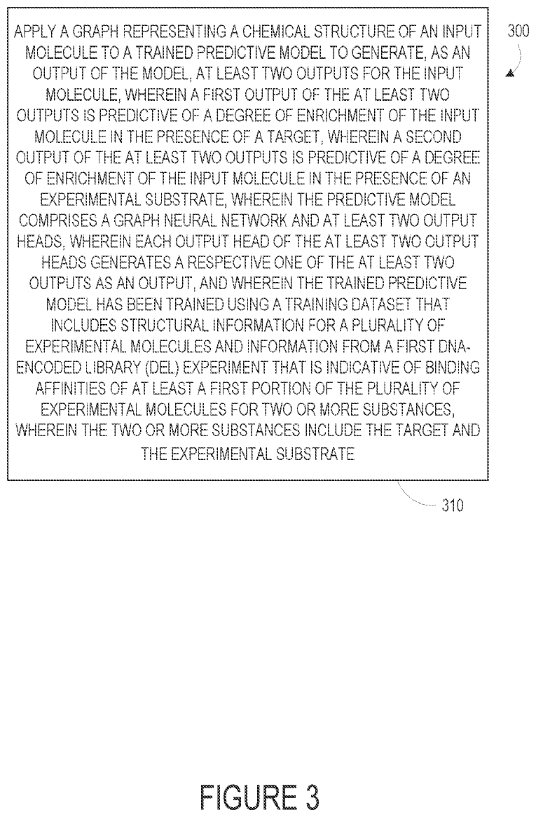

FIG. 3 is a flowchart of an example computer-implemented method 300. The method 300 includes applying a graph representing a chemical structure of an input molecule to a trained predictive model to generate, as an output of the model, at least two outputs for the input molecule, wherein a first output of the at least two outputs is predictive of a degree of enrichment of the input molecule in the presence of a target, wherein a second output of the at least two outputs is predictive of a degree of enrichment of the input molecule in the presence of an experimental substrate, wherein the predictive model comprises a graph neural network and at least two output heads, wherein each output head of the at least two output heads generates a respective one of the at least two outputs as an output, and wherein the trained predictive model has been trained using a training dataset that includes structural information for a plurality of experimental molecules and information from a first DNA-encoded library (DEL) experiment that is indicative of binding affinities of at least a first portion ofthe plurality of experimental molecules for two or more substances, wherein the two or more substances include the target and the experimental substrate (310). The method 300 could include additional or alternative features.

### IV. Example Machine Learning Models and Training Thereof

A machine learning model as described herein may include, but is not limited to: an artificial neural network (e.g., a herein-described neural network, including a graph neural network, convolutional neural network, and/or graph convolutional network, a recurrent neural network, a Bayesian network, a hidden Markov model, a Markov decision process, a logistic regression function, a support vector machine, a suitable statistical machine learning algorithm, and/or a heuristic machine learning system), a support vector machine, a regression tree, an ensemble of regression trees (also referred to as a regression forest), a decision tree, an ensemble of decision trees (also referred to as a decision forest), or some other machine learning model architecture or combination of architectures.

An artificial neural network (ANN) could be configured in a variety of ways. For example, the ANN could include two or more layers, could include units having linear, logarithmic, or otherwise-specified output functions, could include fully or otherwise-connected neurons, could include recurrent and/or feed-forward connections between neurons in different layers, could include filters or other elements to process input information and/or information passing between layers, or could be configured in some other way to facilitate the generation of predicted color palettes based on input images.

An ANN could include one or more filters that could be applied to the input and the outputs of such filters could then be applied to the inputs of one or more neurons of the ANN. For example, such an ANN could be or could include a convolutional neural network (CNN). Convolutional neural networks are a variety of ANNs that are configured to facilitate ANN-based classification or other processing based on molecular structure-encoding graphs or other large-dimensional inputs. An ANN can include a graph neural network (GNN, e.g., a graph convolutional network (GCN)) that is configured to receive a graph as an input, e.g., a graph that is indicative of the molecular structure of a chemical compound (e.g., a small molecule that may be a candidate for a therapeutic clinical intervention).

A GCN or other variety of ANN could include multiple convolutional layers (e.g., corresponding to respective different filters and/or features), pooling layers, rectification layers, fully connected layers, or other types of layers. Rectification layers of a GCN apply a rectifying nonlinear function (e.g., a non-saturating activation function, a sigmoid function) to outputs of a higher layer. Fully connected layers of a GCN receive inputs from many or all of the neurons in one or more higher layers of the GCN. The outputs of neurons of one or more fully connected layers (e.g., a final layer of an ANN or GCN) could be used to determine information about portions or motifs of an input molecular structure (e.g., for each of the atoms of an input structure) or for the molecular structure as a whole.

Neurons in a GCN can be organized according to corresponding dimensions of the input structure. For example, where the input is a structure of a small molecule, neurons of the GCN (e.g., of an input layer of the GCN, of a pooling layer of the GCN) could correspond to locations within the structure of the small molecule (e.g., locations of particular atoms, multi-atomic rings or other structures, etc.). Connections between neurons and/or filters in different layers of the GCN could be related to such locations. For example, a neuron in a convolutional layer of the GCN could receive an input that is based on a convolution of a filter with a portion of the input structure, or with a portion of some other layer of the GCN, that is at a location proximate to the location within the overall molecular structure of the portion of the convolutional-layer neuron. In another example, a neuron in a pooling layer of the CNN could receive inputs from neurons, in a layer higher than the pooling layer (e.g., in a convolutional layer, in a higher pooling layer), that have locations that are proximate to the location of the pooling-layer neuron.

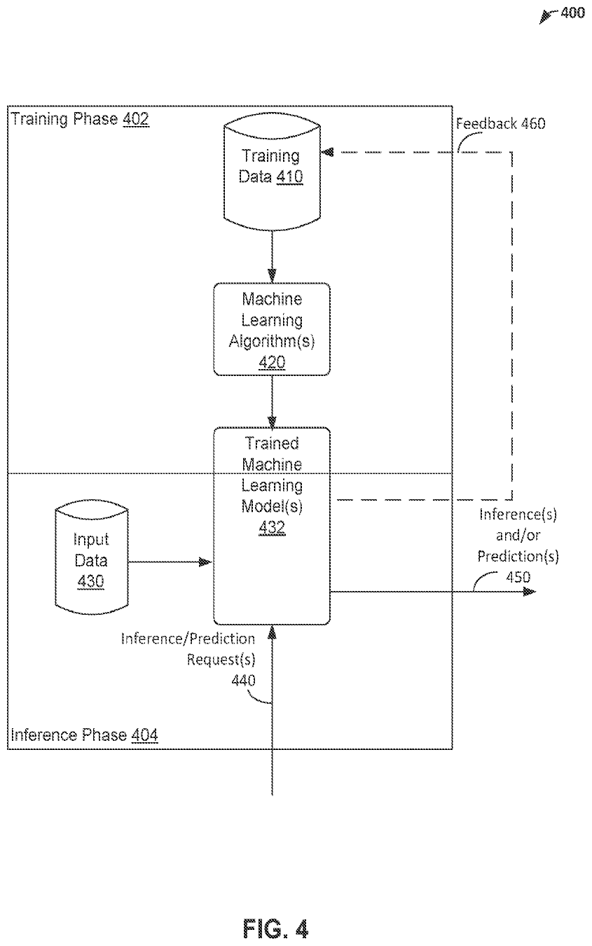

FIG. 4 shows diagram 400 illustrating a training phase 402 and an inference phase 404 of trained machine learning model(s) 432, in accordance with example embodiments. Some machine learning techniques involve training one or more machine learning algorithms, on an input set of training data to recognize patterns in the training data and provide output inferences and/or predictions about (patterns in the) training data. Such output could take the form of experimental data observed that is related to the chemical structure at the input, e.g., DNA sequences, counts, or other DEL experimental data regarding binding affinity of a molecule having the input molecular structure to a target, an experimental substrate, one or more anti-targets, or some other substance(s) of interest. The resulting trained machine learning algorithm can be termed as a trained machine learning model. For example. FIG. 4 shows training phase 402 where one or more machine learning algorithms 420 are being trained on training data 410 to become trained machine learning model 432. Then, during inference phase 404, trained machine learning model 432 can receive input data 430 and one or more inference/prediction requests 440 (perhaps as part of input data 430) and responsively provide as an output one or more inferences and/or predictions 450 (e.g., predicted binding affinities, enrichment levels, or other information that is indicative of a predicted interaction between an input candidate molecular structure and one or more targets, anti-targets, or other substances of interest).

As such, trained machine learning model(s) 432 can include one or more models of one or more machine learning algorithms 420. Machine learning algorithm(s) 420 may include, but are not limited to: an artificial neural network (e.g., a herein-described graph neural network, convolutional network, and/or graph convolutional network, a recurrent neural network, a Bayesian network, a hidden Markov model, a Markov decision process, a logistic regression function, a support vector machine, a suitable statistical machine learning algorithm, and/or a heuristic machine learning system), a support vector machine, a regression tree, an ensemble of regression trees (also referred to as a regression forest), a decision tree, an ensemble of decision trees (also referred to as a decision forest), or some other machine learning model architecture or combination of architectures. Machine learning algorithm(s) 420 may be supervised or unsupervised, and may implement any suitable combination of online and offline learning.

In some examples, machine learning algorithm(s) 420 and/or trained machine learning model(s) 432 can be accelerated using on-device coprocessors, such as graphic processing units (GPUs), tensor processing units (TPUs), digital signal processors (DSPs), and/or application specific integrated circuits (ASICs). Such on-device coprocessors can be used to speed up machine learning algorithm(s) 420 and/or trained machine learning model(s) 432. In some examples, trained machine learning model(s) 432 can be trained, reside and execute to provide inferences on a particular computing device, and/or otherwise can make inferences for the particular computing device.

During training phase 402, machine learning algorithm(s) 420 can be trained by providing at least training data 410 as training input using unsupervised, supervised, semi-supervised, and/or reinforcement learning techniques. Unsupervised learning involves providing a portion (or all) of training data 410 to machine learning algorithm(s) 420 and machine learning algorithm(s) 420 determining one or more output inferences based on the provided portion (or all) of training data 410. Supervised learning involves providing a portion of training data 410 to machine learning algorithm(s) 420, with machine learning algorithm(s) 420 determining one or more output inferences based on the provided portion of training data 410, and the output inference(s) are either accepted or corrected based on correct results associated with training data 410. In some examples, supervised learning of machine learning algorithm(s) 420 can be governed by a set of rules and/or a set of labels for the training input, and the set of rules and/or set of labels may be used to correct inferences of machine learning algorithm(s) 420.

Semi-supervised learning involves having correct results for part, but not all, of training data 410. During semi-supervised learning, supervised learning is used for a portion of training data 410 having correct results, and unsupervised learning is used for a portion of training data 410 not having correct results. Reinforcement learning involves machine learning algorithm(s) 420 receiving a reward signal regarding a prior inference, where the reward signal can be a numerical value. During reinforcement learning, machine learning algorithm(s) 420 can output an inference and receive a reward signal in response, where machine learning algorithm(s) 420 are configured to try to maximize the numerical value of the reward signal. In some examples, reinforcement learning also utilizes a value function that provides a numerical value representing an expected total of the numerical values provided by the reward signal over time. In some examples, machine learning algorithm(s) 420 and/or trained machine learning model(s) 432 can be trained using other machine learning techniques, including but not limited to, incremental learning and curriculum learning.

During inference phase 404, trained machine learning model(s) 432 can receive input data 430 (e.g., input graphs indicative of the chemical structure of candidate small molecule drugs) and generate and output one or more corresponding inferences and/or predictions 450 about input data 430 (e.g., predicted binding affinities, enrichment values, or other information related to the predicted interaction between a molecule having the structure of the input and a target, anti-target, experimental substrate, or other substance of interest). As such, input data 430 can be used as an input to trained machine learning model(s) 432 for providing corresponding inference(s) and/or prediction(s) 450. For example, trained machine learning model(s) 432 can generate inference(s) and/or prediction(s) 450 in response to one or more inference/prediction requests 440. In some examples, trained machine learning model(s) 432 can be executed by a portion of other software. For example, trained machine learning model(s) 432 can be executed by an inference or prediction daemon to be readily available to provide inferences and/or predictions upon request.

### V. Example Implementations and Experimental Data

As noted above, methods described herein train models to predict, based on input chemical structure information (e.g., structure graphs) separate DEL experimental outcomes (e.g., enrichment with respect to a target substance, an experimental substrate, and one or more anti-target substances) separately, rather than predicting a single output label that represents such experimental data in a more complex, experiment-variable, and potentially information-discarding manner. In this way, the labels predicted (and/or used to train such a model) naturally reflect the physical meaning of each selection experiment—the resulting model makes a prediction for each experimental outcome and these predictions can be flexibly combined as needed in downstream applications.

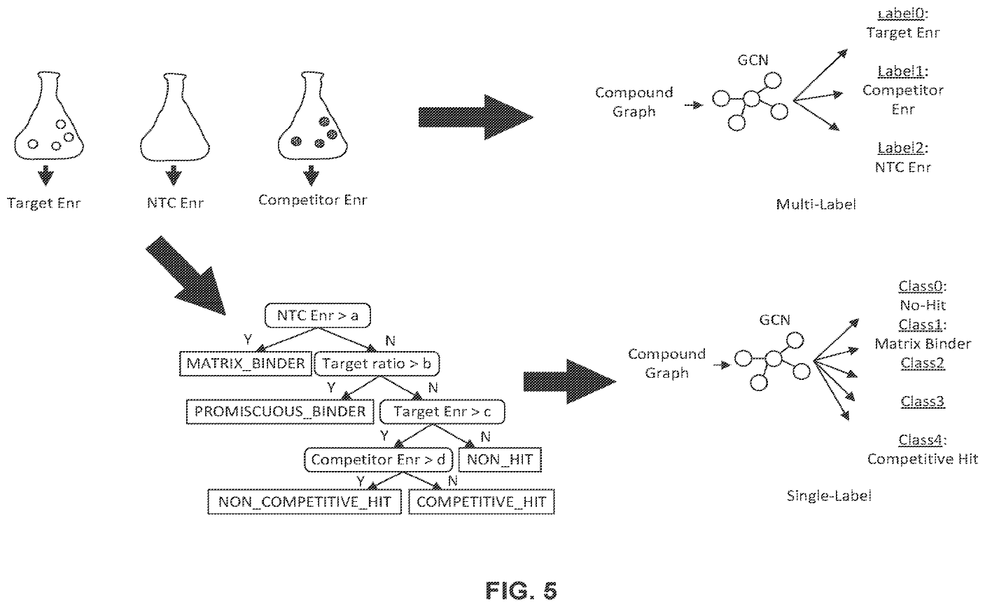

The methods and other embodiments described herein were validated by 1) designing and implementing key components of a multi-label neural architecture as described herein that models DEL data more directly (an example of such being depicted in FIG. 5), and 2) conducting in silico retrospective testing experiments, thereby demonstrating that this technique of modeling each DEL selection experiment separately (e.g., as the output of respective heads of a multi-head machine learning model) can bring better performance than previously attempts to predict a single, complex multi-class label. Additionally, to examine performance in real drug-discovery settings, wet lab prospective testing experiments were performed for two protein targets, demonstrating the superior performance of multi-label models as described herein in hit-finding applications.

To achieve such performance in a multi-label model as described herein, the training data was sampled as follows. Each selection experiment label exhibited a wide range of enrichment scores. Each label was categorized into high enrichment and low enrichment types, and the model was provided during training with equal chances of high enrichment vs low enrichment training examples. So, for example, if 3 output labels were available (i.e., 3 selection experiments to experimentally assess enrichment of candidate molecules against a target, an experimental substrate material, and an anti-target), then the number of all combinatorial types was 8. Each training data batch was balanced with respect to example counts across all these types (e.g., such that the number of examples in the training data batch differed across the types by less than 5%).

Additionally, the multiple prediction scores (one per selection experiment) output from the GCNN multi-label model were combined into a single overall composite score or prediction (e.g., a “reduced label”) and used for checkpoint selection (e.g., by comparing the “reduced label” outputs of the partially-trained models with a validation dataset). Such “reduced labels” can also be used for downstream tasks (e.g., to propose sets of molecules to find hits). Through in-silico evaluation, the final label reduction scheme used was:

$$Reduced label = Target - Enr - label - NTC - Enr - label - Competitor - Enr - label$$

This can be alternatively expressed as the composite score for a particular experimental molecule being determined by subtracting a predicted multi-class label for an experimental substrate and a predicted multi-class label for an anti-target from a predicted multi-class label for the target.

To compare hit-finding performance of the models described herein with the current state-of-the-art GCNN single-label multi-class model, two relatively hard protein targets were selected: Tyrosine-protein kinase (c-KM and Estrogen Receptor Alpha (ERa). In prior work, c-KIT and ERa showed relatively low hit rates (9.7% and 18.8% respectively at concentration of 10 uM), giving the models described herein significant room to improve. Two types of graph neural networks were trained to facilitate direct comparison: a GCNN multi-class single-label model used previously in McCloskey et al., Machine learning on DNA-Encoded libraries: A new paradigm for hit finding. J. Med. Chem., June 2020; and a GCNN multi-label model as described herein developed in this study. FIG. 5 depicts the aspects of this competitive assessment; the outputs of the target enrichment (“Target Enr”), no-target control enrichment (“NTC Enr”), and competitor (or “anti-target”) enrichment (“Competitor Enr”) experiments across a range of chemical compounds (each represented by a “Compound Graph”) are used to train a multi-label model as described herein (upper GCN of FIG. 5) or a single-label model as was used previously (lower GCN of FIG. 5). The experimental enrichment outputs were separately thresholded to generate respective separate output labels for training the multi-label model. The experimental enrichment outputs were applied to a manually-designed ‘column scheme’ (depicted as a structured flow chart of thresholds applied to the enrichment data) to generate a single output label for training the single-label model.

Internal inhibitor datasets were used to perform in silico retrospective evaluation of the multi-label models as described herein and to compare them against state-of-the-art single-label models. Five test datasets were available for ERa and two were available for c-KIT. A metric that is closely related to hit-rate was determined and used for assessment: the number of active molecules among the top 100 highest scoring molecules in the test set (“recall@100”). Table 1 shows the results of these assessments. For six out of seven test sets, the GCNN multi-label models as described herein outperformed the single-label, multi-class counterpart. Table 1 compares a multi-label model as described herein (“GCNN multi-label”) with the single-label, multi-class model of the prior art (“GCNN multiclass”). GCNN multilabel (a) reports the results for the multi-label model as described herein trained using target enrichment directly as the final reduced label (i.e., reduced-label=Target-Enr-label), while GCNN multilabel (b) reports the results for the multi-label model as described herein trained using a composite score as the final reduced label (i.e., Reduced-label=Target-Enr-label−NTC-Enr-label−Competitor-Enr-label).

| actives@100 (↑) | c-KIT-test0 | c-KIT-test1 |
|---|---|---|
| GCNN multiclass | 27 | 59 |
| GCNN multilabel (a) | 28 | 61 |
| GCNN multilabel (b) | 29 | 57 |

| | ERa- | ERa- | ERa- | ERa- | ERa- |
|---|---|---|---|---|---|
| actives@100 (↑) | test0 | test1 | test2 | test3 | test4 |
| GCNN multiclass | 99 | 77 | 42 | 23 | 13 |
| GCNN multilabel (a) | 100 | 79 | 43 | 26 | 11 |
| GCNN multilabel (b) | 100 | 79 | 45 | 26 | 12 |

The training data was preprocessed, including a step of disynthon aggregation to denoise the raw DNA-sequencing count data. During prospective experimental testing, each model type proposed a list of ˜200 molecules from the same commercially purchasable library (Mcule instock library was for the validation experiments described herein) and the percentage of hits was measured in a wet lab. Human intervention in the molecule list selection was limited by automating diversity selection and structural filtering into a streamlined pipeline, so that the hit-rate difference should be mostly explained by model type difference. Simplified relative to a two-step prospective testing approach, single-point inhibition assays were used at the molecule concentration of 10 uM in the validation experiments described herein.

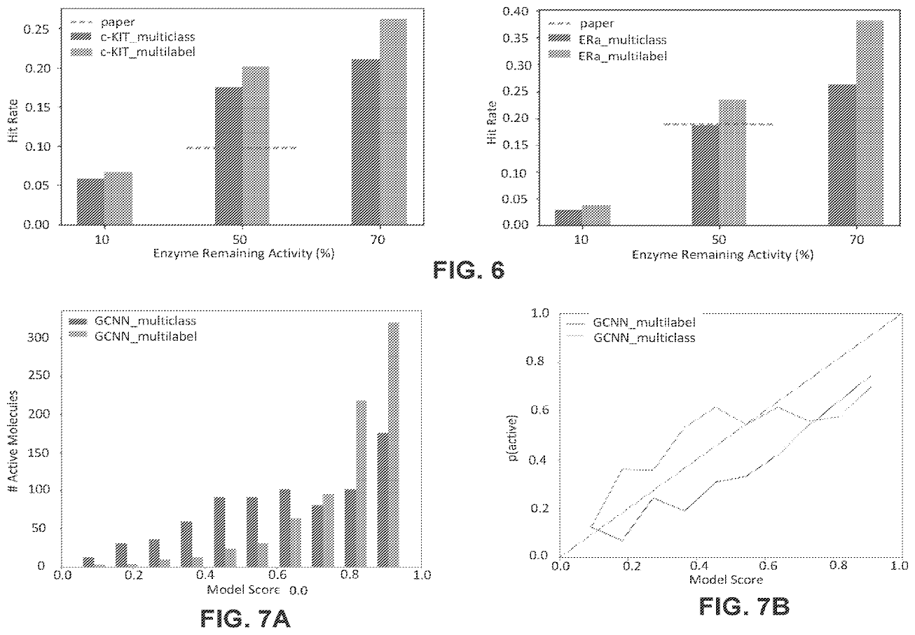

For both of the c-KIT and ERa protein targets, the GCNN multi-label model as described herein outperformed the previous GCNN single-label, multi-class model at three different inhibition cut-off percentages (see FIG. 6). Specifically, for the c-KIT target, at 30% inhibition (enzyme remaining activity=70%), the multi-label model achieved a 26.2% hit rate while the single-label, multi-class model had a hit rate of 20.9%; at 30% inhibition, the multi-label model achieved a 20.1% hit 80 rate while the single-label, multi-class model had a 17.4% hit rate. The hit rates reported previously for the single-label, multi-class model are also included as an additional reference; the difference in results between the single-label, multi-class model as implemented in the present validation study and as reported previously are likely the result of recent improvements in model hyper parameters, differences in the commercial libraries from which molecules were selected, and a difference in diversity and filtering strategies.

FIG. 7A shows the predictive probability distribution of hit molecules of an internal ERa inhibitor dataset using the GCNN multi-class model as described herein and the GCNN single-class, multi-label model used for comparison. FIG. 7B shows a reliability diagram for these two models

Note that, using an internal ERa inhibitor dataset (test1) as an illustrative example, the GCNN single-label, multi-class model placed many active molecules in the middle range of the score spectrum. This could be caused by unrealistic assumptions made in creating the mutually exclusive single labels on which that model was trained. In contrast, GCNN multi-label model tended to score the active molecules (true inhibitors) more towards the high end compared to the GCNN single-label model (see FIG. 7A). FIG. 7B shows that the GCNN multi-label model has an improved calibration which enriches actives in the high score region.

The multi-label model architecture for DEL data modeling provided herein allows models to learn available DEL experiment data more naturally and more directly than the current state-of-the-art approach. The use of such a multi-label model also reduces or eliminates the dependency on manually-specified and thus potentially expensive, subjective, and variable column scheme. The multi-label architecture provides improvements in retrospective test datasets and also in web-lab prospective testing and provides improvements in a hit-finding example use-case. The improved calibration of such multi-label models tends to score active compounds towards higher scores than the previous single-label, multi-class GCNN model that was used as a baseline for comparison.

### VI. Conclusion

The particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

Additionally; while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are contemplated herein.

### VII. Enumerated Example Embodiments

Embodiments of the present disclosure may thus relate to one of the enumerated example embodiments (EEEs) listed below. It will be appreciated that features indicated with respect to one EEE can be combined with other EEEs.

EEE 1 is a computer-implemented method including: (i) obtaining training data for a plurality of experimental molecules, wherein the training data comprises structural information for each experimental molecule in the plurality of experimental molecules and information from a first DNA-encoded library (DEL) experiment that is indicative of binding affinities of at least a first portion of the plurality of experimental molecules for two or more substances, wherein the two or more substances include a target and an experimental substrate; (ii) based on the training data, determining at least two multi-class labels for each experimental molecule in the plurality of experimental molecules, wherein determining at least two multi-class labels for a given experimental molecule of the plurality of experimental molecules comprises (a) determining a first multi-class label that is indicative of a degree of enrichment of the given experimental molecule in the presence of the target, and (b) determining a second multi-class label that is indicative of a degree of enrichment of the given experimental molecule in the presence of the experimental substrate; (iii) applying the training data to train a predictive model to receive, as an input, a graph representing a chemical structure of an input molecule and to generate, as an output, the at least two multi-class labels for the input molecule, wherein the predictive model comprises a graph neural network and at least two output heads, wherein each output head of the at least two output heads generates a respective one of the at least two multi-class labels as an output; and (iv) outputting the trained predictive model.

EEE 2 is the computer-implemented method of EEE 1, wherein determining at least two multi-class labels for the given experimental molecule of the plurality of experimental molecules additionally comprises determining a third multi-class label that is indicative of a degree of enrichment of the given experimental molecule in the presence of an anti-target.

EEE 3 is the computer-implemented method of EEE 2, wherein the anti-target is one of hERG, ERa, or a specified region of the target.

EEE 4 is the computer-implemented method of EEE 1, wherein determining at least two multi-class labels for the given experimental molecule of the plurality of experimental molecules additionally comprises determining a third multi-class label that is indicative of a degree of enrichment of the given experimental molecule in the presence of a first anti-target and determining a fourth multi-class label that is indicative of a degree of enrichment of the given experimental molecule in the presence of a second anti-target.

EEE 5 is the computer-implemented method of EEE 4, wherein the first anti-target is hERG and the second anti-target is ERa.

EEE 6 is the computer-implemented method of any of EEEs 1-5, wherein each multi-class label of the at least two multi-class labels contains two classes.

EEE 7 is the computer-implemented method of any of EEEs 1-6, wherein applying the training data to train the predictive model comprises: (i) applying structural information for a subset of experimental molecules of the plurality of experimental molecules to the predictive model to generate respective predicted sets of the at least two multi-class labels; and (ii) based on the predicted sets of the at least two multi-class labels, generating respective composite scores for each experimental molecule in the subset of experimental molecules.

EEE 8 is the computer-implemented method of EEE 7, wherein applying the training data to train the predictive model additionally comprises: (i) comparing the generated composite scores to a validation dataset; and (ii) based on the comparison, at least one of: (a) selecting a checkpoint for a prospective predictive model, (b) selecting a prospective model from a set of prospective models for folding and/or replication, (c) combining a set of prospective models to generate the predictive model, (d) terminating training of the predictive model.

EEE 9 is the computer-implemented method of EEE 7 or EEE 8, wherein determining at least two multi-class labels for the given experimental molecule of the plurality of experimental molecules additionally comprises determining a third multi-class label that is indicative of a degree of enrichment of the given experimental molecule in the presence of an anti-target, and wherein generating a composite score for a particular experimental molecule in the subset of experimental molecules comprises subtracting a predicted second multi-class label and predicted third multi-class label for the particular experimental molecule from a predicted first multi-class label for the particular experimental molecule.

EEE 10 is the computer-implemented method of any of EEEs 1-9, further comprising: balancing the training data such that each possible multi-label condition represented by the at least two multi-class labels is represented by a respective number of training examples that does not differ across the possible multi-label conditions by more than 5%

EEE 11 is the computer-implemented method of any of EEEs 1-10, further comprising: applying the trained predictive model to generate respective predicted sets of the at least two multi-class labels for a plurality of candidate molecules.

EEE 12 is the computer-implemented method of EEE 11, further comprising: (i) based on the predicted sets of the at least two multi-class labels generated for the plurality of candidate molecules, selecting a subset of the plurality of candidate molecules; and (ii) performing an additional experiment to assess the selected subset of the plurality of candidate molecules.

EEE 13 is the computer-implemented method of EEE 11 or EEE 12, further comprising: based on the predicted sets of the at least two multi-class labels generated for the plurality of candidate molecules, generating respective composite scores for each candidate molecule in the plurality of candidate molecules, wherein selecting a subset of the plurality of candidate molecules based on the predicted sets of the at least two multi-class labels generated for the plurality of candidate molecules comprises selecting the subset of the plurality of candidate molecules based on the composite scores generated for the plurality of candidate molecules.

EEE 14 is the computer-implemented method of any of EEEs 1-13, wherein the training data comprises information from a second DEL experiment that is indicative of binding affinities of at least a second portion of the plurality of experimental molecules for the two or more substances, wherein the second DEL experiment differs from the first DEL experiment.

EEE 15 is a computer-implemented method comprising: applying a graph representing a chemical structure of an input molecule to a trained predictive model to generate, as an output of the model, at least two outputs for the input molecule, wherein a first output of the at least two outputs is predictive of a degree of enrichment of the input molecule in the presence of a target, wherein a second output of the at least two outputs is predictive of a degree of enrichment of the input molecule in the presence of an experimental substrate, wherein the predictive model comprises a graph neural network and at least two output heads, wherein each output head of the at least two output heads generates a respective one of the at least two outputs as an output, and wherein the trained predictive model has been trained using a training dataset that includes structural information for a plurality of experimental molecules and information from a first DNA-encoded library (DEL) experiment that is indicative of binding affinities of at least a first portion of the plurality of experimental molecules for two or more substances, wherein the two or more substances include the target and the experimental substrate.

EEE 16 is the computer-implemented method of EEE 15, wherein generating at least two outputs for the input molecule additionally comprises determining a third output that is predictive of a degree of enrichment of the input molecule in the presence of an anti-target.

EEE 17 is the computer-implemented method of EEE 16, wherein the anti-target is one of hERG, ERa, or a specified region of the target.

EEE 18 is the computer-implemented method of EEE 15, wherein generating at least two outputs for the input molecule additionally comprises determining a third output that is predictive of a degree of enrichment of the input molecule in the presence of a first anti-target and determining a fourth output that is predictive of a degree of enrichment of the input molecule in the presence of a second anti-target.

EEE 19 is the computer-implemented method of EEE 18, wherein the first anti-arget is hERG and the second anti-target is ERa.

EEE 20 is the computer-implemented method of any of EEEs 15-19, further comprising: based on the generated at least two outputs, generating a composite score for the input molecule.

EEE 21 is the computer-implemented method of EEE 20, wherein generating at least two outputs for the input molecule additionally comprises determining a third output that is predictive of a degree of enrichment of the input molecule in the presence of an anti-target, and wherein generating a composite score for the input molecule comprises subtracting the second output and third output from the first output.

EEE 22 is a computing device comprising: one or more processors, wherein the one or more processors are configured to perform the method of any of EEEs 1-21.

EEE 23 is an article of manufacture including a non-transitory computer-readable medium, having stored thereon program instructions that, upon execution by a computing device, cause the computing device to perform operations to effect the method of any of EEEs 1-21.

1. A computer-implemented method comprising:
obtaining training data for a plurality of experimental molecules, wherein the training data comprises structural information for each experimental molecule in the plurality of experimental molecules and information from a first DNA-encoded library (DEL) experiment that is indicative of binding affinities of at least a first portion of the plurality of experimental molecules for two or more substances, wherein the two or more substances include a target and an experimental substrate;
based on the training data, determining at least two multi-class labels for each experimental molecule in the plurality of experimental molecules, wherein determining at least two multi-class labels for a given experimental molecule of the plurality of experimental molecules comprises (i) determining a first multi-class label that is indicative of a degree of enrichment of the given experimental molecule in the presence of the target, and (ii) determining a second multi-class label that is indicative of a degree of enrichment of the given experimental molecule in the presence of the experimental substrate;
applying the training data to train a predictive model to receive, as an input, a graph representing a chemical structure of an input molecule and to generate, as an output, the at least two multi-class labels for the input molecule, wherein the predictive model comprises a graph neural network and at least two output heads, wherein each output head of the at least two output heads generates a respective one of the at least two multi-class labels as an output; and
outputting the trained predictive model.

2. The computer-implemented method of claim 1, wherein determining at least two multi-class labels for the given experimental molecule of the plurality of experimental molecules additionally comprises determining a third multi-class label that is indicative of a degree of enrichment of the given experimental molecule in the presence of an anti-target.

3. The computer-implemented method of claim 2, wherein the anti-target is one of hERG, ERa, or a specified region of the target.

4. The computer-implemented method of claim 1, wherein determining at least two multi-class labels for the given experimental molecule of the plurality of experimental molecules additionally comprises determining a third multi-class label that is indicative of a degree of enrichment of the given experimental molecule in the presence of a first anti-target and determining a fourth multi-class label that is indicative of a degree of enrichment of the given experimental molecule in the presence of a second anti-target.

5. The computer-implemented method of claim 4, wherein the first anti-target is hERG and the second anti-target is ERa.

6. The computer-implemented method of claim 1, wherein each multi-class label of the at least two multi-class labels contains two classes.

7. The computer-implemented method of claim 1, wherein applying the training data to train the predictive model comprises:
applying structural information for a subset of experimental molecules of the plurality of experimental molecules to the predictive model to generate respective predicted sets of the at least two multi-class labels;
based on the predicted sets of the at least two multi-class labels, generating respective composite scores for each experimental molecule in the subset of experimental molecules;
comparing the generated composite scores to a validation dataset; and
based on the comparison, at least one of: (i) selecting a checkpoint for a prospective predictive model, (ii) selecting a prospective model from a set of prospective models for folding and/or replication, (iii) combining a set of prospective models to generate the predictive model, (iv) terminating training of the predictive model.

8. (canceled)

9. The computer-implemented method of claim 7, wherein determining at least two multi-class labels for the given experimental molecule of the plurality of experimental molecules additionally comprises determining a third multi-class label that is indicative of a degree of enrichment of the given experimental molecule in the presence of an anti-target, and wherein generating a composite score for a particular experimental molecule in the subset of experimental molecules comprises subtracting a predicted second multi-class label and predicted third multi-class label for the particular experimental molecule from a predicted first multi-class label for the particular experimental molecule.

10. The computer-implemented method of claim 1, further comprising:
balancing the training data such that each possible multi-label condition represented by the at least two multi-class labels is represented by a respective number of training examples that does not differ across the possible multi-label conditions by more than 5%.

11. The computer-implemented method of claim 1, further comprising:
applying the trained predictive model to generate respective predicted sets of the at least two multi-class labels for a plurality of candidate molecules.

12. The computer-implemented method of claim 11, further comprising:
based on the predicted sets of the at least two multi-class labels generated for the plurality of candidate molecules, selecting a subset of the plurality of candidate molecules; and
performing an additional experiment to assess the selected subset of the plurality of candidate molecules.

13. The computer-implemented method of claim 11, further comprising:
based on the predicted sets of the at least two multi-class labels generated for the plurality of candidate molecules, generating respective composite scores for each candidate molecule in the plurality of candidate molecules,
wherein selecting a subset of the plurality of candidate molecules based on the predicted sets of the at least two multi-class labels generated for the plurality of candidate molecules comprises selecting the subset of the plurality of candidate molecules based on the composite scores generated for the plurality of candidate molecules.

14. The computer-implemented method of claim 1, wherein the training data comprises information from a second DEL experiment that is indicative of binding affinities of at least a second portion of the plurality of experimental molecules for the two or more substances, wherein the second DEL experiment differs from the first DEL experiment.

15. A computer-implemented method comprising:
applying a graph representing a chemical structure of an input molecule to a trained predictive model to generate, as an output of the model, at least two outputs for the input molecule, wherein a first output of the at least two outputs is predictive of a degree of enrichment of the input molecule in the presence of a target, wherein a second output of the at least two outputs is predictive of a degree of enrichment of the input molecule in the presence of an experimental substrate, wherein the predictive model comprises a graph neural network and at least two output heads, wherein each output head of the at least two output heads generates a respective one of the at least two outputs as an output, and wherein the trained predictive model has been trained using a training dataset that includes structural information for a plurality of experimental molecules and information from a first DNA-encoded library (DEL) experiment that is indicative of binding affinities of at least a first portion of the plurality of experimental molecules for two or more substances, wherein the two or more substances include the target and the experimental substrate.

16. The computer-implemented method of claim 15, wherein generating at least two outputs for the input molecule additionally comprises determining a third output that is predictive of a degree of enrichment of the input molecule in the presence of an anti-target.

17. The computer-implemented method of claim 16, wherein the anti-target is one of hERG, ERa, or a specified region of the target.

18. The computer-implemented method of claim 15, wherein generating at least two outputs for the input molecule additionally comprises determining a third output that is predictive of a degree of enrichment of the input molecule in the presence of a first anti-target and determining a fourth output that is predictive of a degree of enrichment of the input molecule in the presence of a second anti-target.

19. The computer-implemented method of claim 18, wherein the first anti-target is hERG and the second anti-target is ERa.

20. (canceled)

21. The computer-implemented method of claim 20, wherein generating at least two outputs for the input molecule additionally comprises determining a third output that is predictive of a degree of enrichment of the input molecule in the presence of an anti-target, and wherein generating a composite score for the input molecule comprises subtracting the second output and third output from the first output.

22. (canceled)

23. An article of manufacture including a non-transitory computer-readable medium, having stored thereon program instructions that, upon execution by a computing device, cause the computing device to perform operations to effect the method of claim 1.
